(19) United States
(12) Patent Application Publication
(10) Pub. No.: US 2025/0032556 A1
(43) Pub. Date: Jan. 30, 2025

(54) GUT BACTERIUM STARIN AND USE THEREOF

(71) Applicant: SUN YAT-SEN UNIVERSITY, Guangzhou (CN)

(72) Inventors: Min XIA, Guangzhou (CN); Yan LIU, Guangzhou (CN); Zhongxia LI, Guangzhou (CN); Ludi LIU, Guangzhou (CN); Yingxi XU, Guangzhou (CN)

(73) Assignee: SUN YAT-SEN UNIVERSITY, Guangzhou (CN)

(21) Appl. No.: 18/917,849

(22) Filed: Oct. 16, 2024

Related U.S. Application Data
(63) Continuation-in-part of application No. PCT/CN2024/078282, filed on Feb. 23, 2024.

(30) Foreign Application Priority Data
Feb. 27, 2023 (CN) 202310169904.6

(51) Int. Cl. A61K 35/741; A61P 19/06

(52) U.S. Cl. CPC A61K 35/741; A61P 19/06

(57) ABSTRACT

Provided are a gut bacterium strain and uses thereof, particularly, the gut bacterium strain with an accession number of GDMCC 62983 (Alistipes indistinctus Xia-1), and a method for the treatment and/or prevention of hyperuricemia using the gut bacterium strain of the present invention. The gut bacterium strain of the present invention has a therapeutic effect on lowering uric acid and a high safety, solving the technical problems of side effects caused by long-term intake of common drugs for hyperuricemia on the market, as well as the poor effect of controlling diet.

### CROSS REFERENCE TO RELATED APPLICATIONS

This application is a continuation-in-part (CIP) application based upon PCT Application No. PCT/CN2024/078282 filed Feb. 23, 2024, which claims the priority of Chinese Patent Application No. 202310169904.6, filed with the China National Intellectual Property Administration on Feb. 27, 2023 and titled with “GUT BACTERIUM STARIN AND USE THEREOF”, the disclosures of which are hereby incorporated by reference in their entirety.

### FIELD

The present disclosure relates to the technical field of gut microorganism, and in particular to a gut bacterium strain and use thereof.

### BACKGROUND

Hyperuricemia (HUA) is a metabolic disease with high incidence and prevalence worldwide that poses a serious health risk. It is estimated that the total prevalence of HUA in China is currently about 13.3% (about 170 million people), of which 19.4% in males is higher than 7.9% in females. Abnormal elevation of serum uric acid not only causes gout, but also leads to major chronic metabolic diseases, such as metabolic syndrome, cardiovascular and cerebrovascular diseases, and chronic kidney disease. Hyperuricemia is a predictive factor for the development of hypertension, metabolic syndrome, type 2 diabetes, coronary artery disease, left ventricular hypertrophy, atrial fibrillation, myocardial infarction, stroke, heart failure and chronic kidney disease. Therefore, it is important to find ways to regulate hyperuricemia/serum uric acid level.

The common methods for managing hyperuricemia are described below.
1. Drug treatment: The main regulatory pathway can be divided into two main categories: reducing the synthesis of uric acid by liver, and enhancing the excretion of uric acid by kidney. Drugs acting through these two pathways have side effects such as liver and kidney damage in long-term use. Allopurinol is prone to cause hypersensitivity reactions, which has a lethality rate of up to 30% once occurred. Febuxostat is not only expensive, but also a potential cardiovascular risk. Benzbromarone has been reported to cause outbreaks of hepatic necrosis in the Caucasian race, and so on.
2. Diet control, which is not very effective. Purine-restricted diet is also one of the common means in the prevention and control of HUA. However, since dietary sources account for only a small portion (about 20%) of the purine sources of uric acid synthesis, while the vast majority comes from endogenous production such as cellular metabolism, the effect of dietary control is not satisfied.

It can be seen that the common drugs for hyperuricemia on the market have side effects when taken for long periods of time, and dietary control is not effective, so there is a need for safe and effective drugs and methods to regulate HUA.

### SUMMARY

In view of this, the present disclosure provides a gut bacterium strain and use thereof, and the gut bacterium strain can promote the excretion of uric acid, thereby lowering the uric acid level in the body.

In the first aspect, the present disclosure provides a gut bacterium strain deposited in Guangdong Microbial Culture Collection Center under an accession number of 62983 (GDMCC 62983).

Deposit information: the gut bacterium strain of the present invention was deposited under accession number GDMCC 62983 in the Guangdong Microbial Culture Collection Center on Nov. 17, 2022, named Alistipes indistinctus Xia-1. The address of GDMCC is: No.59Building, No. 100, Xianlie Zhong Road, Yuexiu District, Guangzhou City, Guangdong Province, China, Postal Code 510070.

In the second aspect, the present disclosure provides use of a gut bacterium strain deposited under GDMCC 62983 in the manufacture of a product for the treatment/prevention of hyperuricemia. The gut bacterium strain has a significant effect on lowering uric acid.

Preferably, the dosage form of the product is a solid, semi-solid or liquid for oral or injection administration.

Preferably, the gut bacterium strain Alistipes indistinctus Xia-1 is used to promote the excretion of uric acid in the body.

More specifically, the gut bacterium strain Alistipes indistinctus Xia-1 is used to promote the excretion of uric acid through intestinal tract in the body.

In the third aspect, the present disclosure provides a product comprising the gut bacterium strain deposited under GDMCC 62983.

Specifically, the product comprises the gut bacterium strain and the bacterium strain after subculture.

Preferably, the form of the gut bacterium strain is selected from the group consisting of a viable bacterium, a dead bacterium, a bacterial cellular component, a bacterial cellular extract, a bacterial cellular lysate, a supernatant from the gut bacterium strain culture, and a combination thereof.

Preferably, the gut bacterium strain in the product is in an amount of 10⁶ to 10¹²cfu/dose.

Specifically, the product further comprises microorganisms such as bacteria and/or fungi; the bacteria may be microorganisms such as probiotics and the fungi may be microorganisms such as yeast.

Preferably, the dosage form of the product is a solid, semi-solid or liquid for oral or injection administration.

Preferably, the product is a pharmaceutical composition, a dietary supplement composition, a nutritional composition, a nutraceutical composition, a food composition, a feed additive composition, a culture, or a combination thereof.

More preferably, the food composition may be a solid beverage, a sweet or fruit juice, a dairy product (e.g. yoghurt, flavored fermented milk, lactobacillus drink, and cheese).

More preferably, the form of the nutritional composition, nutraceutical composition, food composition, feed additive composition and culture may be a liquid, a solid, a suspension or a powder.

More preferably, the dosage form of the pharmaceutical composition and dietary supplement composition is a pill, a powder, a capsule, a tablet, a granule, a capped film, an orally soluble granule, a sachet, a sugar-coated pill or a liquid.

Specifically, the product may be a drug, dietary supplement, nutritional supplement, nutraceutical, general food, feed additives and the like.

More preferably, the culture includes cell-free culture filtrate of Alistipes indistinctusXia-1 and the bacteria after subculture.

Preferably, the product further comprises a pharmaceutically acceptable adjuvant, a food acceptable adjuvant and a metabolite, or a combination thereof.

More preferably, the pharmaceutically acceptable adjuvant is selected from the group consisting of an excipient, a disintegrant, a lubricant, a sweeting agent, a binder, and a combination thereof.

The metabolite is an end product, such as an enzyme produced in metabolism of the gut bacterium strain, a cell structure component, an extracellular polysaccharide, bacteriocin, or a combination thereof.

More preferably, the food acceptable adjuvant is selected from the group consisting of a mineral, a vitamin, a dietary fibre, a prebiotic, a protein (e.g. an enzyme), a carbohydrate, a lipid (e.g. a fat), a plant extract (e.g. a plant extract), an amino acid, an immunomodulator, a milk substitute, and a combination thereof.

Specifically, the pharmaceutically acceptable adjuvant and said food-acceptable adjuvant are existing conventional compositions and are not specifically described in this application.

In the fourth aspect, the present disclosure provides use of the product (comprising Alistipes indistinctus Xia-1 and the bacteria after subculture) in the treatment and/or prevention of hyperuricemia.

Also provided is use of the pharmaceutical composition, dietary supplement composition, nutritional composition, nutraceutical composition, food composition, feed additive composition, and culture in the treatment and/or prevention of hyperuricemia.

Also provided is a method of preventing or treating hyperuricemia comprising administering the gut bacterium strain (Alistipes indistinctus Xia-1) described in the present invention to the subject in need thereof.

In some embodiments, the gut bacterium strain comprises a viable bacterium, a dead bacterium, a bacterial cellular component, a bacterial cellular extract, a bacterial cellular lysate, a supernatant from the gut bacterium strain, or a combination thereof.

In some embodiments, the gut bacterium strain is formulated in a solid, semi-solid or liquid for oral or injection administration.

In some embodiments, the gut bacterium strain is in a form of a pharmaceutical composition, a dietary supplement composition, a nutritional composition, a nutraceutical composition, a food composition, a feed additive composition, a culture, or a combination thereof.

In some embodiments, the gut bacterium strain is formulated with a pharmaceutically acceptable adjuvant, a food acceptable adjuvant, a metabolite of the gut bacterium strain, or a combination thereof. The pharmaceutically acceptable adjuvant may be selected from the group consisting of an excipient, a disintegrant, a lubricant, a sweeting agent, a binder, and a combination thereof. The metabolite may be an enzyme produced in metabolism of the gut bacterium strain, a cell structure component, an extracellular polysaccharide, bacteriocin, or a combination thereof. The food acceptable adjuvant may be a mineral, a vitamin, a dietary fiber, a prebiotic, a protein, a carbohydrate, a lipid, a plant extract, an amino acid, an immunomodulator, a milk substitute, or a combination thereof.

In the past, using a drug or changing lifestyle was usually applied to regulate uric acid level, targeting liver (uric acid synthesis) and kidney (uric acid excretion). But fewer studies have been conducted with the intestinal flora. The main organ that promotes uric acid excretion is the kidney (60-70%), so previous studies have focused on the filtration, reabsorption and secretion mechanisms of uric acid in the kidney. As research continues, more and more studies have identified the gut, which is the organ where the other one-third (30-40%) of SUA is excreted, as an important pathway in uric acid metabolism that has long been overlooked. With the rise of intestinal microecology, this disclosure identifies intestinal excretion as a novel pathway for lowering uric acid.

To solve the technical problem that common hyperuricemia drugs have side effects when taken for a long period of time, and the effect of controlling diet is not obvious, the present disclosure provides a gut bacterium strain deposited under GDMCC 62983. The gut bacterium strain promotes the excretion of uric acid in the body, so that play a role in prevention and/or treatment of hyperuricemia. The present disclosure further provides a product comprising Alistipes indistinctus Xia-1, which can be a drug, dietary supplement, nutritional supplement, nutraceutical, general food, feed additive, etc., enabling organisms (e.g., human beings or animals, etc.) to achieve therapeutic and/or preventive effects on hyperuricemia during treatment or in the daily diet.

### BRIEF DESCRIPTION OF DRAWINGS

FIG. 1 shows a schematic diagram of the animal experiment in the Example of the present disclosure.

FIG. 2 shows the serum uric acid (SUA) levels in different treatment groups in the Example of the present disclosure, wherein, control represents the negative control group, model represents the model group of hyperuricemia, AI represents the intervention group treated with Alistipes indistinctus Xia-1, Ben represents the intervention group treated with benzbromarone, a classical drug for hyperuricemia. The results are expressed as mean+standard error, and*** denotes P<0.001.

FIG. 3 shows the change curves of inosine-induced SUA in different treatment groups in the Example of the present disclosure, wherein, control represents the negative control group, model represents the model group of hyperuricemia, AI represents the intervention group treated with Alistipes indistinctus Xia-1, and Ben represents the intervention group treated with benzbromarone, a classical drug for hyperuricemia.

FIG. 4 shows the area under the curve in different treatment groups of inosine inducement experiment provided in the Example of the present disclosure, wherein control represents the negative control group, model represents the model group of hyperuricemia, AI represents the intervention group treated with Alistipes indistinctus Xia-1, Ben represents the intervention group treated with benzbromarone, a classical drug for hyperuricemia. The results are expressed as mean+standard error, * denotes P<0.05, and **** denotes P<0.0001.

FIG. 5 shows the intestinal excretion level of uric acid in different treatment groups in the Example of the present disclosure, wherein control represents the negative control group, model represents the model group of hyperuricemia, AI represents the intervention group treated with Alistipes indistinctus Xia-1, Ben represents the intervention group treated with benzbromarone, a classical drug for hyperuricemia. The results are expressed as mean+standard error, and * denotes P<0.05.

FIG. 6 shows the renal excretion of uric acid in different treatment groups in the Example of the present disclosure, wherein control represents the negative control group, MODEL represents the model group of hyperuricemia, AI represents the intervention group treated with Alistipes indistinctus Xia-1, Ben represents the intervention group treated with benzbromarone, a classical drug for hyperuricemia. The results are expressed as mean+standard error, and * denotes P<0.05.

### DETAILED DESCRIPTION

The present disclosure provides a gut bacterium strain and use thereof, which effectively solves the technical defects in the prior art that common drugs for hyperuricemia have side effects if taken for a long time, and the dietary control has no obvious effect.

Technical solutions of the present disclosure are clearly and completely described below. Apparently, the embodiments described in the following are only some embodiments of the present disclosure, rather than all the embodiments. Any other embodiments obtained based on the embodiments of the present disclosure by those skilled in the art without any creative effort fall within the scope of protection of the present disclosure.

The gut bacterium strain Alistipes indistinctus Xia-1 used in the following Example was isolated by the inventor and deposited under GDMCC 62983 in Guangdong Microbial Culture Collection Center.

In the following Example, control represents the blank control group, model represents the hyperuricemia model group, AI represents the intervention group (on hyperuricemia model) treated with the gut bacterium strain of Alistipes indistinctus Xia-1 of the present disclosure, and Ben represents the intervention group (on hyperuricemia model) treated with the positive drug benzbromarone.

Alistipes indistinctus Xia-1 strain used in the following Example may be prepared into a solid, semi-solid or liquid for oral or injection administration, for example, powders, tablets, capsules, liquid preparations such as an oral liquid for oral or injection administration.

An adjuvant may be used in the product for the treatment/prevention hyperuricemia, which may be is a pharmaceutically acceptable carrier, diluent or excipient, or an additive complying with food regulations.

### Example 1 Isolation and Culture of Alistipes indistinctus Xia-1

I. Isolation of Alistipes indistinctus Xia-1 Strain
1. Fecal samples were collected from healthy subjects (27-35 years old) and immediately transferred under anaerobic conditions. Initial processing of the samples and subsequent weighing and dilution were also performed under strictly anaerobic conditions.
2. The samples were weighed and diluted to 104, 10-5 and 106 with pre-reduced 0.1 M PBS (0.145M NaCl, 0.15 M sodium phosphate, pH=7), and plated on modified GAM agar (Gifu anaerobic medium agar) supplemented with ox-bile, NaCl, or antibiotics to isolate the target intestinal microorganisms.

The ingredients of modified GAM agar are as follows (preparation volume is 1 liter): peptone, 5.0 g; soy peptone, 3.0 g; protein glycoprotein peptone, 5.0 g; serum powder, 5.0 g; soy peptone, 3.0 g; protein glycoprotein peptone, 5.0 g; serum powder, 10.0 g; yeast extract, 2.5 g; beef extract powder, 2.2 g; liver extract powder, 1.2 g; glucose, 0.5 g; soluble starch, 5.0 g; L-cysteine monohydrochloride 0.3 g; sodium thioglycolate, 0.3 g; potassium dihydrogen phosphate, 2.5 g; sodium chloride, 3.0 g; L-tryptophan, 0.2 g; L-arginine, 1.0 g; vitamin K 1,5 mg; hemin chloride, 10 mg; agar, 15.0 g.
3. The plates were incubated in an anaerobic incubator containing 88% nitrogen, 7% hydrogen and 5% carbon dioxide at 37° C. for 3 days.
4. The strains were isolated using modified GAM agar plates supplemented with norfloxacin (40 mg/ml; Sigma).
5. Pick a single colony and continue to isolate and culture until a monoclonal colony is obtained on modified GAM agar.
6. Single colonies were picked and added to PCR system (40 μl system: 20 μl phanta high-fidelity enzyme, 1.6 μl upstream primer, 1.6 ul downstream primer, 1.6 ul sterile water, 0.8 μl DNA (the DNA from the single colony)). Both the upstream primer and the downstream primer are 16 s universal primers commonly used in the art. The fragments amplified by PCR were sequenced, and the results obtained by sequencing were aligned using the blastn program (from NCBI). Standard databases-Nucleotide collection (nr/nt) was selected to confirm the strain type.
7. The obtained strain was identified as belonging to Alistipes indistinctus and named Alistipes indistinctus Xia-1 strain.
   II. Culture Conditions of Alistipes indistinctus Xia-1 Strain
1. Anaerobic environment: 5% hydrogen+5% carbon dioxide+90% nitrogen
2. Culture temperature: 37° C.
3. Culture time: about 12-48 h
4. Solid culture medium: Columbia Blood Agar Plate, which is available on the market, for example, Huankai Microbial Co. Ltd, Guangdong, China, with the catalog number of CP0160.
5. Liquid culture medium: PYG (Peptone Yeast extract Glucose) medium, which is available on the market, for example, Coolaber Co.Ltd, Beijing, China, with the catalog number of PM0610.
6. Suitable pH: about 7.2
7. The strain reaches the logarithmic growth phase about 14 hours after recovery and reaches the stationary phase at about 38 hours.

### Example 2 Treating Hyperuricemia Using Alistipes indistinctus Xia-1 in Mouse Model

Animal experiments were performed in Example 2 to validate the effects of Alistipes indistinctus Xia-1 on treatment of hyperuricemia.
1. Randomized grouping and establishment of hyperuricemia model

Increasing the source of uric acid or decreasing the excretion of uric acid can elevate the serum uric acid concentration, therefore, in this experiment, the method of administrating uric acid (UA) in current combination with the uricase inhibitor oxalic acid (OA) was chosen, i.e., an animal model of hyperuricemia was constructed by feeding feed supplemented with 2% UA+4% OA.

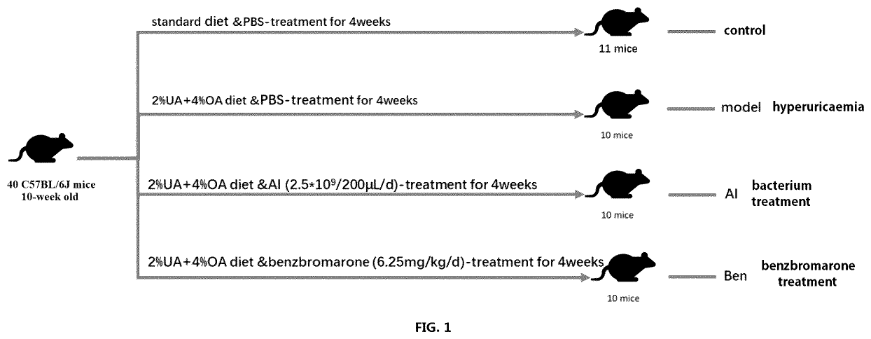

A total of 40 healthy male C57BL/6J mice of 10-week-old (initial weight of about 25 g) were fed adaptively for one week in the laboratory, and then randomly divided into four groups according to body weight, including negative control group (control), hyperuricemia model group (model), Alistipes indistinctus Xia-1 intervention group (AI), and benzbromarone (a classical drug for hyperuricemia) intervention group (Ben), with 10 mice in each group. As shown in FIG. 1, mice in the negative control group were fed with a standard diet for 4 weeks, and mice in the other three groups were fed with diets containing uric acid and uricase inhibitor (2% UA+4% OA by mass fraction) for 4 weeks. Throughout the feeding process, the mice in the AI intervention group was administered orally with Alistipes indistinctus Xia-1 daily at 2.5×10⁹viable bacteria/200 μuL by gavage, the mice in the benzbromarone group was administered with benzbromarone solution at 6.25 mg/kg/day by gavage, and the mice in the remaining two groups were administered orally with an equal amount of sterile PBS (200 μL) once a day for 4 weeks. After intervention, blood samples were taken from the tail vein of mice and centrifuged at 3500rpm for 15 min at low temperature (4° C.). The serum in the upper layer was taken, and the serum uric acid level was detected by using Nanjing Jiancheng Uric Acid Assay Kit.
2. Determination of total excretion of uric acid in mice

Inosine, also known as hypoxanthine nucleoside, is the precursor of uric acid. The uric acid level in blood of mice can be increased by giving inosine via gavage. The uric acid excretion capacity of mice can be evaluated by inosine-stimulated test, thereby speculating that whether the tested substances for lowering uric acid play a role in the inhibition of uric acid synthesis or the promotion of uric acid excretion. After 4 weeks of intervention, the inosine inducement assay was performed to determine the total uric acid excretion capacity of mice in each group. Specifically, blood samples of mice were taken in the morning (8:00-8:30), noted as 0 min, followed by administration of an inosine solution (187.5 mg/kg) via gavage. Blood samples were collected from the tail vein at 15, 30, 60 and 120 min after administration. Each blood sample was centrifuged, and the supernatant was taken and subjected the detection of the uric acid concentration. The change curve of serum uric acid in mice 2 h after administration of inosine was plotted, and the area under the curve (AUC) was used to compare the total uric acid excretory capacity of mice in each group.
3. Determination of intestinal excretion level of uric acid in mice

In order to analyze the effect of Alistipes indistinctus Xia-1 on intestinal excretion of uric acid, this experiment was performed to determine whether the increase in uric acid excretion is through intestinal excretion. After 4-week intervention, mice were anaesthetized by intraperitoneal injection of 10% chloral hydrate (0.34 ml/100 g), and polyethylene tubes were inserted in the superior segment of the duodenum and the middle jejunum, to that formed intestinal loop in the upper part of the small intestine. After removing the intestinal contents by slow infusion of saline, the exocytosis buffer (saline containing 0.3 mM potassium oxonate) was introduced into the intestinal loop, and both ends of the loop were closed with a syringe. The buffer in the loop was collected with syringe every 20 min within 2 h and the uric acid concentration was quantified. The intestinal excretion level of uric acid was calculated by the following formula.

$$Intestinal excretion level of uric acid = ( C \times V \times L 1 ) / L 2.$$

C represents the concentration of urate in the circulation, V represents the volume of buffer solution in the circulation, L1 represents the length of the whole small intestine, and L2represents the length of the small intestinal involving circulation.

After intervention with Alistipes indistinctus Xia-1, compared to the control group, the rate of intestinal excretion of uric acid (slope) was significantly increased, indicating that the intervention with Alistipes indistinctus Xia-1 promoted intestinal excretion of uric acid.
4. Determination of renal excretion level of uric acid in mice

At 24 h after intervention, urine samples were collected from mice using a metabolic cage, and the volume was measured. The collected urine sample was centrifuged at 3000 rpm at low temperature (4° C.) for 5 min, then urinary uric acid (UUA), urinary creatinine (Scr), blood creatinine (Scr), blood urea nitrogen (BUN) were detected in accordance with the instructions of the Nanjing Jiancheng kit, and the renal excretion level of uric acid was calculated by the following formula.

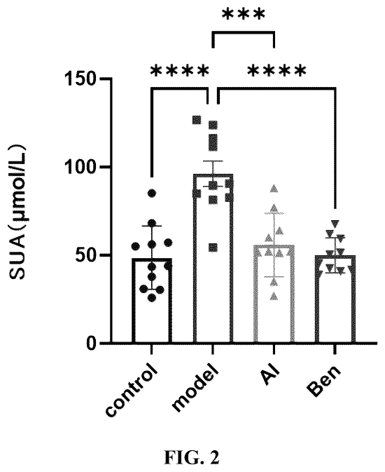

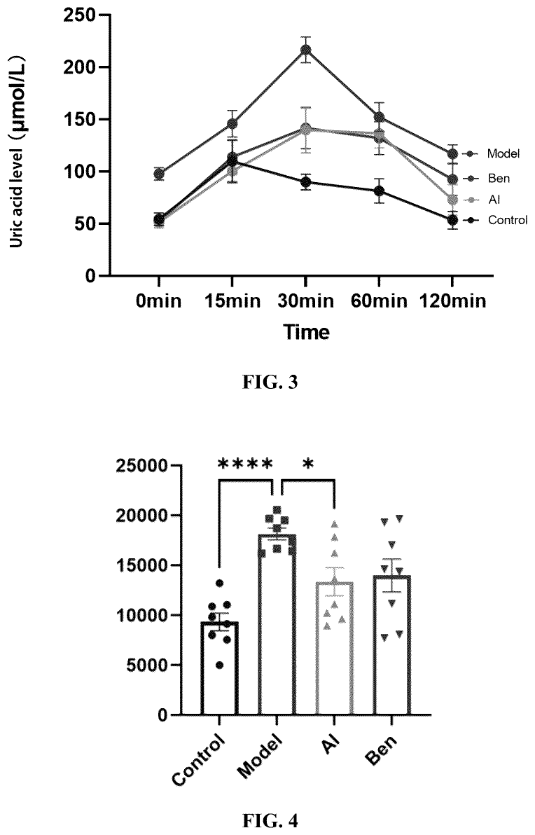

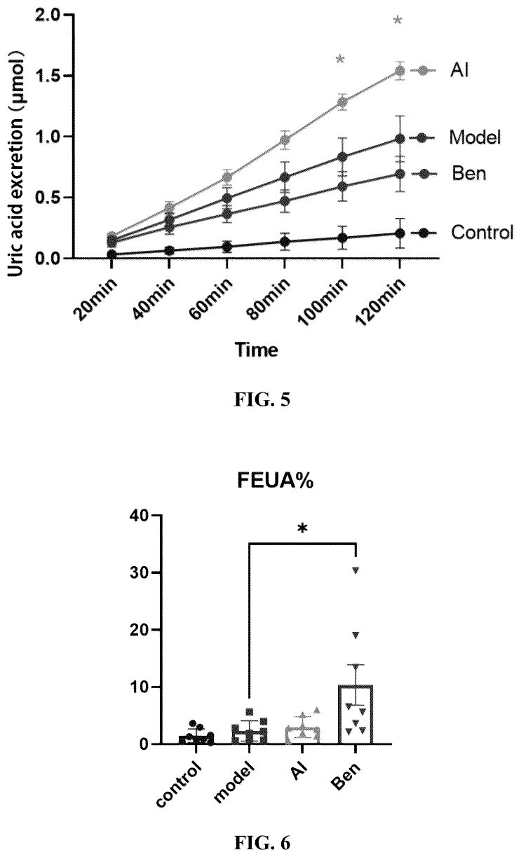

Fractional excretion of uric acid (FEUA %)=UUA×SCr/(SUA×UCr)×100%. This experiment can be used to determine whether a decrease in uric acid levels is associated with an increase in renal excretion. The FEUA % did not change significantly after the intervention with Alistipes indistinctus Xia-1. The benzbromarone acts by targeting renal excretion, so the FEUA % increased significantly after the intervention compared to the model group.
5. The results of the above experiments are shown in FIG. 2-6.

It could be concluded from FIG. 2-6 that,
1. The uric acid level in blood of the model group (model) was significantly higher than that of the normal control group (control), p<0.0001, indicating the success of modeling hyperuricemia. Changes in body weight and food intake of mice were monitored weekly, and it was found that there were no significant differences in body weight, food intake, activity status and other physical signs among the three groups of mice fed with high uric acid. Compared to the model group, Alistipes indistinctus Xia-1 can effectively reduce the weight loss caused by the feed for modeling, and the mice were in good condition throughout the intervention, indicating that the safety of Alistipes indistinctus Xia-1 is good.
2. The uric acid level in blood of mice in AI intervention group (AI) was significantly lower than that in the model group (model), p<0.0001, indicating that the bacterium strain can significantly reduce the uric acid level in blood of hyperuricemic mice and have an effect of lowering uric acid.
3. The uric acid level in blood of mice in benzbromarone (a classic drug for hyperuricemia) intervention group (Ben) was significantly lower than that in the model group (model), p<0.001, indicating that the positive drug benzbromarone can also significantly reduce the uric acid level in blood of hyperuricemic mice, with uric acid-lowering function. This result also indicates that the model is reliable.
4. There is no significant difference in the uric acid level in blood between the AI intervention group (AI) and benzbromarone intervention group (Ben), p>0.05, indicating that the effect of Alistipes indistinctus Xia-1 strain on reducing the uric acid level in blood of hyperuricemic mice is comparable to benzbromarone.

In summary, the present invention found that Alistipes indistinctus Xia-1 1 has the function of lowering serum uric acid level in mice of hyperuricemia model (effect/function study). Alistipes indistinctus Xia-1 strain lowers uric acid level by promoting uric acid excretion, particularly intestinal excretion of uric acid (mechanism study), thus lowering uric acid in the body, showing preventive or/and therapeutic effects on hyperuricemia.

The gut bacterium strain and use thereof provided by the present disclosure are described in detail above. The principle and implementation of the present disclosure are illustrated by using specific embodiments herein. The above descriptions of the embodiments are only used to facilitate understanding of the method and the core idea of the present disclosure. It should be noted that, several improvements and modifications may be made by those skilled in the art to the present disclosure without departing from the principle of the present disclosure, and these improvements and modifications also fall within the protection scope of the claims of the present disclosure.

1. A method of preventing or treating hyperuricemia comprising administering a gut bacterium strain with an accession number of GDMCC 62983 (Alistipes indistinctus Xia-1) to the subject in need thereof.

2. The method according to claim 1, wherein the gut bacterium strain comprises a viable bacterium, a dead bacterium, a bacterial cellular component, a bacterial cellular extract, a bacterial cellular lysate, a supernatant from the gut bacterium strain, or a combination thereof.

3. The method according to claim 1, wherein the gut bacterium strain is formulated in a solid, semi-solid or liquid for oral or injection administration.

4. The method according to claim 1, wherein the gut bacterium strain is in a form of a pharmaceutical composition, a dietary supplement composition, a nutritional composition, a nutraceutical composition, a food composition, a feed additive composition, a culture, or a combination thereof.

5. The method according to claim 1, wherein the gut bacterium strain is formulated with a pharmaceutically acceptable adjuvant, a food acceptable adjuvant, a metabolite of the gut bacterium strain, or a combination thereof.

6. The method according to claim 5, wherein the pharmaceutically acceptable adjuvant is selected from the group consisting of an excipient, a disintegrant, a lubricant, a sweeting agent, a binder, and a combination thereof.

7. The method according to claim 5, wherein the metabolite is an enzyme produced in metabolism of the gut bacterium strain, a cell structure component, an extracellular polysaccharide, bacteriocin, or a combination thereof.

8. The method according to claim 5, wherein the food acceptable adjuvant is a mineral, a vitamin, a dietary fibre, a prebiotic, a protein, a carbohydrate, a lipid, a plant extract, an amino acid, an immunomodulator, a milk substitute, or a combination thereof.
